(19) United States
(12) Patent Application Publication
(10) Pub. No.: US 2002/0010112 A1
(43) Pub. Date: Jan. 24, 2002

(54) Aqueous or aqueous-alcoholic body cleansing compositions comprising sorbitol esters

(75) Inventors: Torsten Henning, Kelkheim (DE); Vera Mulitze-Kleinheyer, Frankfurt am Main (DE)

Correspondence Address: Clariant Corporation, Industrial Property Department, 4331 Chesapeake Drive, Charlotte, NC, 28216, US

(73) Assignee: Clariant GmbH

(21) Appl. No.: 09/798,985

(22) Filed: Mar. 5, 2001

(30) Foreign Application Priority Data
Mar. 4, 2000 (DE) 100 10 768.0

(51) Int. Cl. A61K 7/48; A61K 7/50

(52) U.S. Cl. 510/130; 510/159

(57) ABSTRACT

Aqueous or aqueous-alcoholic body cleansing compositions comprising sorbitol esters obtained by transesterification of optionally oxalkylated sorbitol with fatty acid methyl esters or fatty acid triglycerides and optional oxalkylation of the reaction products obtained by transesterification with fatty acid methyl esters. The sorbitol esters serve as solubilizing agents of active ingredients, as stabilizer, and as care and refatting components.

The invention relates to aqueous or aqueous-alcoholic body cleansing and body care compositions for the mild cleansing and care of the skin, which are notable for a content of sorbitol esters obtained by transesterification of optionally oxalkylated sorbitol with fatty acid methyl esters or fatty acid triglycerides and optional oxalkylation of the reaction products obtained by transesterification with fatty acid methyl esters.

Body cleansing and body care in two steps is time consuming, meaning that compositions with a cleaning and care action at the same time are preferred by many consumers.

A large number of cosmetic products attempts to satisfy this demand.

U.S. Pat. No. 5,612,307 claims aqueous, liquid body cleansing compositions which, in addition to customary surfactant systems, comprise a care component from the group of silicone oils, fats, oils, waxes, hydrophobic plant extracts, fatty acids, alcohols, esters, lipids and/or phospholipids.

WO 94/03152 claims shower gels consisting essentially of a surfactant, silicone oil and a cationic polymer.

It is unsatisfactory that care and moisture-donating components cannot be incorporated in a sufficient amount into aqueous cleanser formulations. A further problem is that aqueous dispersions comprising surfactant systems and moisture-donating and care components separate over the course of time and are thus not very storage-stable.

DE 197 27 950 and DE 199 06 368 describe emulsifiers obtained by transesterification of optionally oxalkylated sorbitol with fatty acid methyl esters or fatty acid triglycerides or oxalkylation of the reaction products obtained by transesterification with fatty acid methyl esters.

The object of the invention was therefore to develop novel skin care body cleansing compositions which combine cleansing and care action and which are free from the abovementioned disadvantages.

The invention provides aqueous or aqueous-alcoholic body cleansing compositions comprising sorbitol esters obtained by transesterification of optionally oxalkylated sorbitol with fatty acid methyl esters or fatty acid triglycerides and optional oxalkylation of the reaction products obtained by transesterification with fatty acid methyl esters.

The sorbitol esters used according to the invention in aqueous or aqueous-alcoholic cosmetic compositions are preferably obtained by a reaction of sorbitol with fatty acid methyl esters or fatty acid triglycerides in accordance with the process described in DE 197 27 950, the catalysts used here being, however, customary alkaline catalysts, in particular sodium methoxide. The fatty acid radical in the fatty acid methyl esters and fatty acid triglycerides generally contains 8 to 22 carbon atoms and can be straight-chain or branched, saturated or unsaturated. Examples thereof are palmitic acid, stearic acid, lauric acid, linoleic acid, linolenic acid, isostearic acid or oleic acid. Suitable fatty acid triglycerides are all native animal or vegetable oils, fats and waxes, for example olive oil, rapeseed oil, palm kernel oil, sunflower oil, coconut oil, linseed oil, castor oil, soybean oil, optionally also in refined or hydrogenated form. Since these natural fats, oils and waxes are normally mixtures of fatty acids having a different chain length, this also applies for the fatty acid radicals in the sorbitol esters used according to the invention.

The reaction of sorbitol with the fatty acid triglycerides or methyl esters is carried out in a one-pot process without solvents at temperatures of approximately 120-140° C. in the presence of an alkaline catalyst. The reaction time is generally 12 to 13 hours. If fatty acid methyl esters are used, the methanol which forms is distilled off during this reaction. Since sorbitol is usually commercially available as an aqueous solution, it is necessary to firstly remove the water. This is effected by distillation at a maximum of 120° C. under reduced pressure. The molar ratio of sorbitol to fatty acid methyl ester is generally 1:1 to 1:2. If fatty acid triglycerides are used, the molar ratio is generally 1 mol of sorbitol per 1 to 4.5 mol, preferably 3.5 to 4.5 mol, of fatty acid triglyceride.

The reaction products may also be alkoxylated, preferably ethoxylated, and the content of ethoxylate groups can be 1 to 90 —CH₂CH₂O— groups per molecule of sorbitol. The alkoxylate groups can be introduced as a result of an alkoxylation of the sorbitol by processes known per se prior to the transesterification reaction. In the case of the transesterification with fatty acid methyl esters, preference is, however, given to a process in which the transesterification is firstly carried out and then, in accordance with processes known per se, the alkoxylate groups are introduced.

In addition to comprising residual amounts of unreacted sorbitol, the reaction product of this transesterification reaction essentially consists of the sorbitol mono fatty acid esters and the sorbitol difatty acid esters. The corresponding triesters are only formed in minor amounts. If fatty acid triglycerides are used as starting material, the reaction product also comprises mono- and difatty acid glyceride and unreacted triglyceride depending on the molar ratio of the starting compounds chosen in each case.

The resulting mixture of the various reaction products is highly suitable as a solubilizing agent of active ingredients, and also as a stabilizer and effects an excellent refatting effect in cosmetic and pharmaceutical compositions.

The amount of the transesterification products according to the invention as refatting, solubilizing and stabilizing agents in aqueous and aqueous-alcoholic compositions is generally 0.1 to 50% by weight, preferably 0.5 to 10% by weight, in particular 1 to 4% by weight, based on the finished composition.

The body cleansing and care compositions according to the invention are not in the form of emulsions, they are, for example, shampoos, shower preparations, shower gels, foam baths, comprising the abovementioned sorbitol esters or mixtures of different sorbitol esters. They can be combined with all customary anionic, cationic, zwitterionic, nonionic and amphoteric surfactants in aqueous or aqueous-alcoholic medium.

The total amount of the surfactants used in the compositions according to the invention can be between 5 and 70% by weight, preferably between 10 and 40% by weight, particularly preferably between 12 and 35% by weight, based on the finished composition.

Anionic washing-active substances which may be mentioned are: C₁₀-C₂₀-alkyl and alkylene carboxylates, alkyl ether carboxylates, fatty alcohol sulfates, fatty alcohol ether sulfates, alkylamide sulfates and sulfonates, fatty acid alkylamide polyglycol ether sulfates, alkane sulfates, alkane sulfonates and hydroxyalkane sulfonates, olefinsulfonates, acyl esters of isothionates, a-sulfo fatty acid esters, alkylbenzenesulfonates, alkylphenol glycol ether sulfonates, sulfosuccinates, sulfosuccinic half-esters and diesters, fatty alcohol ether phosphates, protein/fatty acid condensation products, alkyl monoglyceride sulfates and sulfonates, alkyl glyceride ether sulfonates, fatty acid methyl taurides, fatty acid sarcosinates, sulforicinoleates, amphoacetates or amphoglycinates, acyl glutamates. These compounds and mixtures thereof are used in the form of their water-soluble or water-dispersible salts, for example the sodium, potassium, magnesium, ammonium, mono-, di- and triethanolammonium, and analogous alkylammonium salts.

The proportion by weight of the anionic surfactants in the compositions according to the invention is in the range from 0.1% to 50%, preferably 7 to 30%, particularly preferably 9 to 18%.

Suitable cationic surfactants are, for example, quaternary ammonium salts, such as di-(C₁₀-C₂₄-alkyl)dimethylammonium chloride or bromide, preferably di-(C₁₂-C₁₈-alkyl)dimethylammonium chloride or bromide; C₁₀-C₂₄-alkyldimethylethylammonium chloride or bromide; C₁₀-C₂₄-alkyltrimethylammonium chloride or bromide, preferably cetyltrimethylammonium chloride or bromide and C₂₀-C₂₂-alkyltrimethylammonium chloride or bromide; C₁₀-C₂₄-alkyldimethylbenzylammonium chloride or bromide, preferably C₁₂-C₁₈-alkyldimethylbenzylammonium chloride; N-(C₁₀-C₁₈-alkyl)pyridinium chloride or bromide, preferably N-(C₁₂-C₁₆-alkyl)pyridinium chloride or bromide; N-(C₁₀-C₁₈-alkyl)isoquinolinium chloride, bromide or monoalkyl sulfate; N-(C₁₂-C₁₈-alkylpolyolaminoformylmethyl)pyridinium chloride; N-(C₁₂-C₁₈-alkyl)-N-methylmorpholinium chloride, bromide or monoalkyl sulfate; N-(C₁₂-C₁₈-alkyl)-N-ethylmorpholinium chloride, bromide or monoalkyl sulfate; C1₆-C₁₈-alkylpentaoxethylammonium chloride; diisobutylphenoxyethoxyethyldimethylbenzylammonium chloride; salts of N,N-diethylaminoethylstearylamide and oleylamide with hydrochloric acid, acetic acid, lactic acid, citric acid, phosphoric acid; N-acylaminoethyl-N,N-diethyl-N-methylammonium chloride, bromide or monoalkyl sulfate and N-acylaminoethyl-N,N-diethyl-N-benzylammonium chloride, bromide or monoalkyl sulfate, where acyl is preferably stearyl or oleyl.

The proportion by weight of the cationic surfactants in the compositions according to the invention is in the range from 1 to 10%, preferably 2 to 7%, particularly preferably 3 to 5%.

Examples of suitable nonionic surfactants which can be used as washing-active substances are: fatty alcohol ethoxylates (alkylpolyethylene glycols); alkylphenol polyethylene glycols; alkyl mercaptan polyethylene glycols; fatty amine ethoxylates (alkylaminopolyethylene glycols); fatty acid ethoxylates (acylpolyethylene glycols); polypropylene glycol ethoxylates (Pluronics®); fatty acid amide polyethylene glycol; N-alkyl, N-alkoxypolyhydroxy fatty acid amides, in particular fatty acid N-methylglucamides, sucrose esters; polyglycol ethers, alkyl polyglycosides, phosphoric esters (mono-, di- and triphosphoric esters ethoxylated and nonethoxylated).

The proportion by weight of the nonionic surfactants in the compositions according to the invention is in the range from 1 to 20%, preferably 2 to 10%, particularly preferably 3 to 7%.

Preferred amphoteric surfactants are: N-(C₁₂-C₁₈-alkyl)-β-aminopropionates and N-(C₁₂-C₁₈-alkyl)-p-iminodipropionates as alkali metal and mono-, di- and trialkylammonium salts; N-acylaminoalkyl-N,N-dimethylacetobetaine, preferably N-(C₈-Ci₈-acyl)aminopropyl-N,N-dimethylacetobetaine; C₁₂-C₁₈-alkyldimethylsulfopropylbetaine; amphoteric surfactants based on imidazoline (trade name: Miranol®, Steinapon®), preferably the sodium salt of 1-(β-carboxymethyloxyethyl)-1-(carboxymethyl)-2-laurylimidazolinium; amine oxides, e.g. C₁₂-C₁₈-alkyldimethylamine oxide, fatty acid amidoalkyldimethylamine oxide.

The proportion by weight of the amphoteric surfactants in the compositions according to the invention is in the range from 0.5 to 20%, preferably 1 to 10%.

Furthermore, foam-boosting cosurfactants from the group of alkylbetaines, alkylamidobetaines, aminopropionates, aminoglycinates, imidazoliniumbetaines and sulfobetaines, amine oxides and fatty acid alkanolamides or polyhydroxyamides may be used in the compositions according to the invention.

Preferred surfactants in the compositions according to the invention are lauryl sulfate, cocoamidopropylbetaine, sodium cocoyl glutamate, disodium laureth sulfosuccinate and coconut fatty acid diethanolamide.

In addition, the preparations according to the invention can comprise further additives customary in cosmetics, such as superfatting agents, stabilizers, biogenic active ingredients, glycerol, preservatives, pearlizing agents, dyes and fragrances, solvents, opacifiers, thickeners and dispersants, and also protein derivatives, such as gelatin, collagen-hydrolysates, natural- and synthetic-based polypeptides, egg yolk, lecithin, lanolin and lanolin derivatives, fatty alcohols, silicones, deodorizing agents, substances having a keratolytic and keratoplastic action, enzymes and carrier substances, and also moisture-donating substances. Furthermore, antimicrobially effective agents may be added to the compositions according to the invention.

Superfatty agents which may be used are substances such as, for example, polyethoxylated lanolin derivatives, lecithin derivatives, polyol fatty acid esters, monoglycerides and fatty acid alkanolamides, the latter also serving as foam stabilizers. Typical examples of fats are glycerides, and suitable waxes are, inter alia, beeswax, paraffin wax or microcrystalline waxes, optionally in combination with hydrophilic waxes, e.g. cetylstearyl alcohol.

Stabilizers which can be used are metal salts of fatty acids, such as, e.g. magnesium, aluminum and/or zinc stearate.

Biogenic active ingredients are understood as meaning, for example, plant extracts and vitamin complexes.

Examples of suitable preservatives are phenoxyethanol, formaldehyde solution, parabens, pentanediol or sorbic acid.

Dyes which may be used are the substances approved and suitable for cosmetic purposes.

Suitable thickeners are sodium chloride, potassium chloride, ammonium chloride, sodium sulfate, fatty acid alkylolamides, cellulose derivatives, for example hydroxyethylcellulose, guar gum, polyvinyl alcohol, polyvinylpyrrolidone, hydroxypropyl guar gum, starch and starch derivatives, and natural gums, carboxyvinyl polymers, for example Carbopol® 934, 940, 941, 956, 980, 981, 1342, 1382.

Particularly suitable thickeners and dispersants are ethylene glycol esters of fatty acids having 14 to 22, particularly preferably 16 to 22, carbon atoms, in particular mono- and diethylene glycol stearate. Preference is also given to stearin monoethanolamide, stearin diethanolamide, stearin isopropanolamide, stearin monoethanolamide stearate, stearyl stearate, cetyl palmitate, glyceryl stearate, stearamide diethanolamide distearate, stearamide monoethanolamide stearate, N,N-dihydrocarbyl (C₁₂-C₂₂, in particular C₁₆-C₁₈)-amidobenzoic acid and soluble salts thereof, N,N-di(C₁₆-C₁₈)amidobenzoic acid and derivatives.

The dispersants are used in concentrations of from 0.5 to 10% by weight, preferably from 0.5 to 5% by weight, particularly preferably from 1 to 4% by weight, based on the finished composition.

The desired viscosity of the compositions can be established by adding water and/or organic solvents or by adding a combination of organic solvents and thickeners. In principle, suitable organic solvents are all mono- or polyhydric alcohols and ethoxylated alcohols. Preference is given to using alcohols having I to 4 carbon atoms, such as ethanol, propanol, isopropanol, n-butanol and isobutanol, glycerol and mixtures of said alcohols. Further preferred alcohols are polyethylene glycols having a relative molecular mass below 2000. In particular, a use of polyethylene glycol having a relative molecular mass between 200 and 600 and in amounts up to 45% by weight, and of polyethylene glycol having a relative molecular mass between 400 and 600 in amounts of from 0.5 to 15% by weight is preferred. Further suitable solvents are, for example, triacetin (glycerol triacetate) and 1 -methoxy-2-propanol. The compositions according to the invention can comprise alcohols and ethoxylated alcohols in the amounts by weight of from 0.1% to 50%.

Suitable carrier materials are vegetable oils, natural and hydrogenated oils, waxes, fats, water, alcohols, polyols, glycerol, glycerides, liquid paraffins, liquid fatty alcohols, stearol, polyethylene glycols, cellulose and cellulose derivatives.

Fungicidal active ingredients which may be used are ketoconazole, oxiconazole, bifonazole, butoconazole, cloconazole, clotrimazole, econazole, enilconazole, fenticonazole, isoconazole, miconazole, sulconazole, tioconazole, fluconazole, itraconazole, terconazole, naftifine and terbinafine, Zn pyrethione and octopyrox.

Deodorizing substances which may be used are allantoin and bisabolol in the amounts by weight of from 0.0001 to 10%.

In order to improve the affinity of the sorbitol ester(s) used according to the invention on the skin, it is possible to use cationic guar polymers in the amounts by weight of 0.01 to 1.0%, preferably 0.02 to 0.4%, as described in WO 97/26854.

Suitable cationic polymers are, for example, cationic cellulose derivatives, cationic starch, copolymers of diallylammonium salts and acrylamides, quaternized vinylpyrrolidone/vinylimidazole polymers, condensation products of polyglycols and amines, quaternized collagen polypeptides, quaternized wheat polypeptides, polyethylenimines, cationic silicone polymers, such as e.g. amidomethicones, copolymers of adipic acid and dimethylaminohydroxypropyidiethylenetriamine, polyaminopolyamide and cationic chitin derivatives, such as, for example, chitosan.

Suitable silicone compounds are, for example, dimethylpolysiloxane, methylphenylpolysiloxanes, cyclic silicones, and amino-, fatty-acid-, alcohol-, polyether-, epoxy-, fluorine- and/or alkyl-modified silicone compounds, and also polyalkylsiloxanes, polyalkylarylsiloxanes, polyether siloxane copolymers, as described in U.S. Pat. No. 5,104,645 and publications cited therein, which may, at room temperature, be either in liquid form or in resin form.

The compositions according to the invention can be mixed with conventional ceramides, pseudoceramides, fatty acid N-alkylpolyhydroxyalkylamides, cholesterol, cholesterol fatty acid esters, fatty acids, triglycerides, cerebrosides, phospholipids and similar substances.

Suitable pearlescence-imparting compounds are fatty acid monoalkanolamides, fatty acid dialkanolamides, monoesters or diesters of alkylene glycol, in particular of ethylene glycol and/or propylene glycol or oligomers thereof with higher fatty acids, e.g. palmitic acid, stearic acid or behenic acid or mixtures thereof. Also suitable are mono- or diesters of alkylene glycols with fatty acids, fatty acids and metal salts thereof, monoesters or polyesters of glycerol with carboxylic acids and ketosulfones of a different type. In the compositions according to the invention, particularly preferred pearlescence-imparting components are ethylene glycol distearate and polyethylene glycol distearate having 3 glycol units.

Examples of available moisture-donating substances are isopropyl palmitate, glycerol and/or sorbitol, which can be used in the amounts by weight of 0.1 to 50%.

The compositions according to the invention are prepared in a manner known per se by combination of the individual components and, if required, a further processing adapted to the respective type of preparation. Some of these diverse possible preparation forms are described by way of example in the working examples. With the use according to the invention of sorbitol esters in aqueous or aqueous-alcoholic body cleansing compositions, in particular in shower preparations, shower gels and foam baths, it is possible to avoid damage to the skin and to improve the mildness to the skin of the compositions.

The examples below serve to illustrate the subject-matter of the invention in more detail, without limiting it thereto.

### EXAMPLES

### Example 1

### Shower Gel

| | Component | % by weight |
|---|---|---|
| 1 | Carbopol ETD 2020 | 1.5 |
| 2 | Polyquaternium-10 | 0.3 |
| 3 | Glycerol | 2.0 |
| 4 | Emulsogen SRO | 2.0 |
| 5 | Genagen LDA | 9.2 |
| 6 | Genagen CAB | 4.0 |
| 7 | Hostapon CLG | 4.8 |
| 8 | Citric acid | 0.5 |
| 9 | Methyldibromoglutaronitrile/phenoxyethanol | 0.05 |
| 10 | Perfume | 0.5 |
| 11 | Opacifier Opacifyer 641 | 0.8 |
| 12 | Demineralized water | ad 100 |

Components 1 and 2 were initially introduced and dissolved in about 70° C-hot demineralized water with stirring. 3, 4, 5, 6 and 7 were added successively with stirring, and the pH was adjusted to pH 6.0 with citric acid. By adding 9 and 10, the composition was preserved and perfumed, and then provided with the opacifier 11.

### Example 2

### Shower Gel

| | Component | % by weight |
|---|---|---|
| 1 | Carbopol ETD 2020 | 3.0 |
| 2 | Polyquaternium-10 | 0.3 |
| 3 | Emulsogen SRO | 3.0 |
| 4 | Medialan LD | 2.0 |
| 5 | Genagen LAA | 7.2 |
| 6 | Genagen CAB | 4.0 |
| 7 | Hostapon KOG | 6.9 |
| 8 | Lactic acid | 0.5 |
| 9 | Preservative | q.s. |
| 10 | Perfume | q.s. |
| 11 | Genapol TSM | 1,0 |
| 12 | Demineralized water | ad 100 |

Components 1 and 2 were initially introduced and dissolved in about 70° C.-hot demineralized water with stirring. 3, 4, 5, 6 and 7 were added successively with stirring, and the pH was adjusted to pH 6.0 with lactic acid. By adding 9 and 10, the composition was preserved and perfumed, and then provided with the pearlizing agent 11.

List of Products Used

| | | |
|---|---|---|
| ® Carbopol ETD 2020 | (Clariant GmbH) | Polyacrylic acid, crosslinked |
| SRO ® Emulsogen | (Clariant GmbH) | Sorbitol ester based on rapeseed oil |
| ® Genagen LDA | (Clariant GmbH) | Lauryl amphodiacetate, Na salt |
| ® Genagen LAA | (Clariant GmbH) | Lauryl amphoacetate, Na salt |
| ® Genagen CAB | (Clariant GmbH) | Cocamidopropylbetaine |
| ® Hostapon CLG | (Clariant GmbH) | Sodium lauryl glutamate |
| ® Hostapon KCG | (Clariant GmbH) | Sodium cocoyl glutamate |
| ® Medialan LD | (Clariant GmbH) | Sodium lauroyl sarcosinate |
| ® Genapol TSM | (Clariant GmbH) | PEG-3 distearate, sodium laureth sulfate |
| ® Opacifier 641 | | Na methacrylate/styrene copolymer |

The product Emulsogen SRO (sorbitol ester of rapeseed oil) was prepared as follows:

1 mol of sorbitol in the form of a 70% strength sorbitol syrup was initially introduced and the water was distilled off at 120° C. in a water-pump vacuum. Following the addition of 1% by weight (based on the total initial weight) of potassium carbonate (30% strength in water at 80° C.), the water was distilled off under a complete water-pump vacuum. 4 mol of refined rapeseed oil were then added, and the mixture was stirred for 8 hours at 140° C.

1. An aqueous or aqueous-alcoholic body cleansing composition comprising sorbitol esters obtained by transesterification of optionally oxalkylated sorbitol with fatty acid methyl esters or fatty acid triglycerides and optional oxalkylation of the reaction products obtained by transesterification with fatty acid methyl esters.

2. The body cleansing composition as claimed in claim 1, which comprises sorbitol esters obtained by transesterification of sorbitol with fatty acid triglycerides.

3. The body cleansing composition as claimed in claim 1, which comprises sorbitol esters obtained by reaction of 1 mol of sorbitol with 3.5 to 4.5 mol of fatty acid triglyceride.

4. The body cleansing composition as claimed in claim 1, which comprises 0.1 to 50% by weight of sorbitol esters.
